(19) United States
(12) Patent Application Publication
(10) Pub. No.: US 2026/0224902 A1
(43) Pub. Date: Aug. 6, 2026

(54) EXTRACARDIAC EVOKED-RESPONSE SENSING

(71) Applicant: Medtronic, Inc., Minneapolis, MN (US)

(72) Inventors: Matthew J. Hoffman, St. Paul, MN (US); Hank T. Bink, Golden Valley, MN (US); Vladimir P. Nikolski, San Diego, CA (US); William J. Clemens, Fridley, MN (US); Lars M. Mattison, Blaine, MN (US)

(21) Appl. No.: 19/150,432

(22) PCT Filed: Jan. 17, 2024
(86) PCT No.: PCT/IB2024/050459

§ 371 Date: Jul. 23, 2025

Related U.S. Application Data
(60) Provisional application No. 63/481,350, filed on Jan. 24, 2023.

(51) Int. Cl. A61N 1/37; A61B 5/395; A61N 1/04; A61N 1/05; A61N 1/32

(52) U.S. Cl. CPC A61N 1/371; A61B 5/395; A61N 1/0404; A61N 1/05; A61N 1/327

(57) ABSTRACT

A stimulation device is configured to be coupled to an extracardiac elongated structure that includes a set of electrodes. The stimulation device includes stimulation circuitry, sensing circuitry, and processing circuitry. The processing circuitry is configured to: control the stimulation circuitry to deliver, based on a set of stimulation parameters, a set of stimulation pulses to the target site; control the sensing circuitry to sense a set of evoked response signals from the target site, wherein each evoked response signal of the set of evoked response signals is in response to a corresponding stimulation pulse from the set of stimulation pulses; measure a set of evoked response parameters for the set of evoked response signals; and determine, based on the set of stimulation parameters and the set of evoked response parameters, a likelihood of sensation at the target site from a pacing therapy.

This application claims the benefit of U.S. Provisional Patent Application Ser. No. 63/481,350, filed Jan. 24, 2023, the entire content of which is incorporated herein by reference.

### TECHNICAL FIELD

This disclosure relates generally to cardiac therapy and, more particularly, to devices configured to deliver cardiac pacing and detect extracardiac stimulation.

### BACKGROUND

Medical device systems have been devised to provide electrical stimulation therapy without placing implantable medical leads within the heart or attaching implantable medical leads directly to the heart. These medical device systems may provide, for example, bradycardia pacing, anti-tachyarrhythmia pacing (ATP), post-shock pacing or other types of pacing to the heart from a non-transvenous or non-intracardiac location, such as from a location outside of the heart. In some patients, the medical device system implanted within the patient may also provide cardioversion or defibrillation therapy to the heart of the patient to terminate certain types of tachyarrhythmias, such as ventricular tachycardia (VT) or ventricular fibrillation (VF) to prevent sudden cardiac death (SCD).

### SUMMARY

Medical device systems, such as implantable medical device systems or partially implantable medical device systems, configured to provide electrical stimulation therapy using electrodes outside of the heart may result in the patient experiencing sensation (e.g., paresthesia, pain, etc.) during the delivered stimulation. In the case of an implantable medical device (IMD) system configured to deliver pacing therapy to the heart of a patient, e.g., bradycardia pacing, anti-tachyarrhythmia pacing (ATP), post-shock pacing, pause prevention pacing, cardiac resynchronization therapy (CRT) pacing, or other types of pacing, from an extracardiac location, stimulation of skeletal muscles and intercostal nerves (and/or any other muscle tissue and nerve tissue) may occur proximate the electrodes of the lead or device delivering the therapy.

In accordance with techniques of this disclosure, a stimulation device may process a set of evoked response signals to determine a likelihood of sensation during pacing therapy at a specific implant location. As used herein, the term “evoked response” may refer to the electrical signal from any excitable tissues (including but not limited to neural or muscle tissue) that can be observed by sensing electrodes after electrical stimulation. In other words, an evoked response may be a potential measurement of the reaction of surrounding tissues to a pacing stimulus. The evoked response may be sensed neural or sensed muscle (EMG) activity. In general, one or more parameters of a set of evoked response signals may be correlated with sensation. Accordingly, the stimulation device may evaluate the dependence of evoked response signal parameters on stimulation parameters to determine, for example, proximity to tissues of interest, changes in the tissue, a disruption in conduction, the presence or development of a durable lesion, etc.

In some examples, a stimulation device comprises: stimulation circuitry; sensing circuitry; and processing circuitry configured to: control the stimulation circuitry to deliver, based on a set of stimulation parameters, a set of stimulation pulses to a target site; control the sensing circuitry to sense a set of evoked response signals from the target site, wherein each evoked response signal of the set of evoked response signals is in response to a corresponding stimulation pulse from the set of stimulation pulses; measure a set of evoked response parameters for the set of evoked response signals; and determine, based on the set of stimulation parameters and the set of evoked response parameters, a likelihood of sensation at the target site from a pacing therapy.

In some examples, a system comprises: an extracardiac elongated structure configured to be navigated from an access point of a patient to a target site within a patient, wherein a distal portion of the elongated structure comprises a set of electrodes; and a stimulation device configured to be coupled to the extracardiac elongated structure, wherein the stimulation device comprises: stimulation circuitry; sensing circuitry; and processing circuitry configured to: control the stimulation circuitry to deliver, based on a set of stimulation parameters, a set of stimulation pulses to the target site; control the sensing circuitry to sense, based on a set of sensing parameters, a set of evoked response signals from the target site, wherein each evoked response signal of the set of evoked response signals is in response to a corresponding stimulation pulse from the set of stimulation pulses; measure a set of evoked response parameters for the set of evoked response signals; and determine, based on the set of stimulation parameters and the set of evoked response parameters, a likelihood of sensation at the target site from a pacing therapy.

In some examples, a method comprises: delivering a set of stimulation pulses to the target site based on a set of stimulation parameters; sensing a set of evoked response signals from the target site, wherein each evoked response signal of the set of evoked response signals is in response to a corresponding stimulation pulse from the set of stimulation pulses; measuring a set of evoked response parameters for the set of evoked response signals; and determining, based on the set of stimulation parameters and the set of evoked response parameters, a likelihood of sensation at the target site from a pacing therapy.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual diagram illustrating an example medical system in accordance with techniques of this disclosure.

FIG. 2 is a conceptual diagram illustrating an example lead in accordance with techniques of this disclosure.

FIG. 3 is a block diagram illustrating an example configuration of a stimulation device in accordance with techniques of this disclosure.

FIG. 4 is a block diagram illustrating an example configuration of a stimulation device in accordance with techniques of this disclosure.

FIG. 5 is a conceptual diagram illustrating an example implantable medical system comprising an external electrode in accordance with techniques of this disclosure.

FIG. 6A is a chart illustrating an example set of evoked response signals produced by tissue prior to an irreversible electroporation procedure in accordance with techniques of this disclosure.

FIG. 6B is a chart illustrating an example set of evoked response signals produced by tissue following an irreversible electroporation procedure in accordance with techniques of this disclosure.

FIGS. 7A and 7B are charts illustrating example sets of evoked response signals produced by tissue prior to and following an irreversible electroporation procedure in accordance with techniques of this disclosure.

FIG. 8 is a flow diagram of an example technique for using a medical system in accordance with techniques of this disclosure.

FIG. 9 is a flow diagram of an example technique for using a medical system in accordance with techniques of this disclosure.

FIG. 10 is a flow diagram of an example technique for using a medical system in accordance with techniques of this disclosure.

### DETAILED DESCRIPTION

In general, electrical stimulation of body tissue and organs may be used as a method of treating various conditions. Such stimulation is generally delivered by means of electrical contact between an implantable medical device (IMD) and a target site via one or more electrodes, such as stimulation electrodes disposed on implantable medical electrical leads connected to the IMD and/or electrodes located on the IMD. Examples of IMDs may include implantable pacemakers and implantable cardioverter defibrillators (ICDs), including extravascular implantable pacemakers and extravascular implantable cardioverter defibrillators (EV-ICDs), and cardiac resynchronization therapy (CRT) devices. Examples of IMD may also include devices having electrodes for stimulation disposed on both the lead and housing (e.g., can) of the IMD. It will be appreciated that the techniques of this disclosure may also be applicable to devices that do not have leads, e.g., a leadless pacemaker within the substernal space or some other location.

Although the techniques of this disclosure are described in the context of IMDs, the techniques may also be utilized in partially implantable medical device systems, such as temporary or external medical device systems having a pulse generator outside of the body of a patient coupled to one or more medical electrical leads that are implanted at least partially within the patient. Additionally, the techniques of this disclosure may be useful for applications other than cardiac applications, such as vagus nerve stimulation, AV-nodal stimulation (extracardiac or endocardial), splanchnic nerve stimulation, phrenic nerve stimulation, or other neuromodulation applications. For example, the techniques of this disclosure may use evoked bulk neural activity (e.g., electrical compound action potential-ECAPs) or other signals of interest (e.g., including signals from the phrenic nerve) to determine a likelihood of sensation.

IMDs may deliver cardiac pacing and/or anti-tachyarrhythmia shocks via one or more electrodes of the leads. In general, a patient may experience sensation (e.g., paresthesia, pain, etc.) during pacing due to, for example, stimulation of skeletal muscles and intercostal nerves (and/or any other muscle tissue and nerve tissue) proximate the electrodes of the leads.

In accordance with techniques of this disclosure, a stimulation device, such as an IMD, may process a set of evoked response signals to determine a likelihood of sensation during pacing therapy at a specific implant location and/or stimulation electrode configuration or vector. In general, one or more parameters (e.g., latency, morphology, sensing vector, frequency spectra, evoked response amplitude, etc.) of a set of evoked response signals may be correlated with sensation. Accordingly, the stimulation device may evaluate the dependence of evoked response signal parameters on stimulation parameters (e.g., polarity, pulse width, pulse frequency, stimulation amplitude, stimulation vector, etc.) to determine, for example, likelihood of sensation or pain associated with the pacing stimulation, proximity to tissues of interest, changes in the tissue, a disruption in conduction, the presence or development of a durable lesion, etc.

The techniques of this disclosure may be implemented perioperatively (e.g., around the time of surgery or during surgery) to evaluate the quality of an implant location and/or evaluated stimulation vectors, or during ambulatory use to potentially select a different stimulation vector, titrate therapy (e.g., adjust the stimulation parameters, such as amplitude, pulse width, stimulation vector, etc.) below the level of sensation, etc. In some examples, the techniques may be used to evaluate the status of an incapacitation procedure such as such as radiofrequency (RF) ablation, cryoablation, irreversible electroporation, etc. Thus, the techniques may enable evaluating (e.g., perioperatively) the likelihood of sensation for a particular stimulation vector, set of stimulation parameters, and electrode placement. The techniques may further enable the evaluation of the efficacy of interventions to incapacitate targeted tissues (e.g., via a variety of mechanisms for addressing sensation including ablation, Botox, paralytics, etc.), and the mitigation of similar concerns.

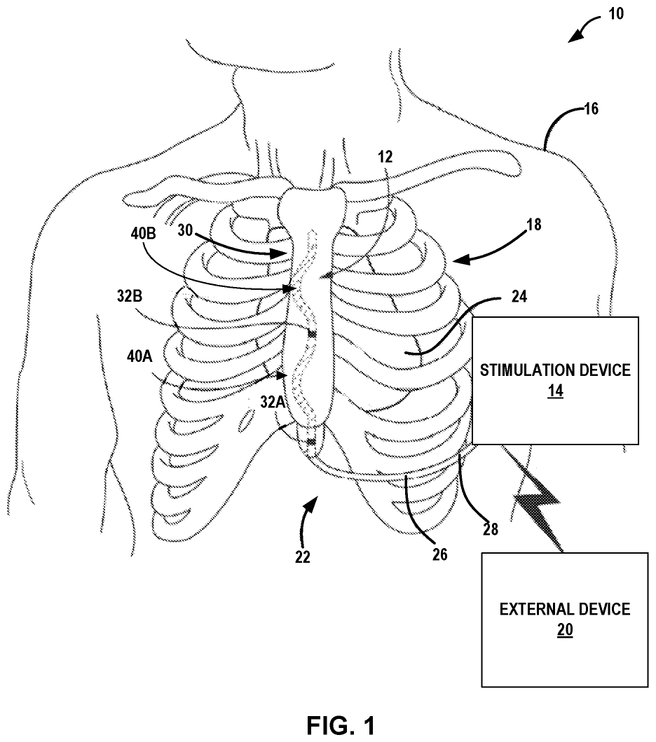

FIG. 1 is a conceptual diagram of an example medical system 10 (“system 10”) in accordance with techniques of this disclosure. System 10 is primarily described herein as an extravascular and/or extracardiac medical system, such as an EV-ICD system with a lead placed between the sternum 12 and the pericardial surface, a subcutaneous system with lead placed extra-thoracically outside of the ribcage, an intrapericardial system with the lead placed within pericardium, an epicardial system with the lead attached to the epicardial surface of the heart, or a pleural system with the lead placed within the pulmonary pleural space. However, it should be understood that the techniques of this disclosure may apply to other medical device systems, such as intravascular and/or intracardiac medical systems, without limitation. Additionally, it should be understood that the techniques of this disclosure may apply to non-cardiac devices (e.g., neurostimulators, pelvic and gastric devices, etc.). Thus, in general, the techniques of this disclosure may apply to any medical device or system that delivers electrical therapy that may cause unintended sensation.

System 10 may include a stimulation device 14. Stimulation device 14 may include a signal generator configured to provide cardiac pacing and/or defibrillation therapy. Stimulation device 14 may be an implantable medical device (IMD) configured to be implanted subcutaneously within the patient. In the example of FIG. 1, stimulation device 14 is implanted subcutaneously on the left mid-axillary of a patient 16, superficially of the patient's ribcage 18. In some examples, stimulation device 14 may be an external device. For instance, stimulation device 14 may be an external pacemaker that is configured to be worn by or carried by a patient. In other instances, stimulation device 14 may be an external device that is used during an implantation procedure for an implantable or partially implantable system. Examples of stimulation device 14 may further include a cardiac resynchronization therapy (CRT) device, a neurostimulator, etc.

Stimulation device 14 may be configured to be coupled to an extracardiac elongated structure. The extracardiac elongated structure is primarily described herein as an implantable medical lead 22 (“lead 22”). However, it should be understood that the extracardiac elongated structure may be an introducer, an implant tool, an ablation catheter, a mapping catheter or other device that is inserted into the body of the patient during a procedure, and that the techniques of this disclosure may apply equally in those examples as well.

Lead 22 may be configured to be navigated from an access point of patient 16 to a target site (which may or may not be extracardiac) within patient 16. Lead 22 may include a lead body 26 sized to be implanted extra-thoracically (outside the ribcage and sternum, e.g., subcutaneously or submuscularly) or intra-thoracically (e.g., beneath the ribcage or sternum, sometimes referred to as a “substernal” position) proximate a heart 24 of patient 16. For example, lead 22 may extend subcutaneously toward the center of the torso of patient 16 and toward the xiphoid process of patient 16.

At least a portion of a body 26 of lead 22 (“lead body 26”) may have a generally undulating shape or pattern (e.g., zig-zag, meandering, sinusoidal, serpentine, or other pattern). Additionally or alternatively, lead body 26 may have a generally uniform shape along the length of lead body 26. In another configuration, lead body 26 may have a flat, ribbon, or paddle shape along at least a portion of the length of the lead body 26.

Other lead body 26 designs may be used without departing from the scope of this application. Lead body 26 of lead 22 may be formed from a non-conductive material, including silicone, polyurethane, fluoropolymers, mixtures thereof, and other appropriate materials, and shaped to form one or more lumens (not shown), however, the techniques are not limited to such constructions.

Lead body 26 may include a proximal portion 28 and a distal portion 30. Distal portion 30 may include a set of electrodes configured to deliver electrical energy to the heart or sense electrical energy within the heart. As used herein, a set may refer to one or more elements. Thus, a set of electrodes may refer to one or more electrodes. Distal portion 30 may be anchored to a desired position within the patient, for example, substernally or subcutaneously by, for example, suturing distal portion 30 to the patient's musculature, tissue, or bone at the xiphoid process entry site. Alternatively, distal portion 30 may be anchored to the patient or through the use of a fixation mechanism, such as rigid tines, prongs, barbs, clips, screws, flanges, etc. For example, distal portion 30 may be anchored proximate a target site within patient 16.

In some examples, distal portion 30 of lead body 26 may be implanted within the anterior mediastinum. The anterior mediastinum may be viewed as being bounded laterally by the pleurae, posteriorly by the pericardium, and anteriorly by sternum 12. In some instances, the anterior wall of the anterior mediastinum may also be formed by the transversus thoracis and one or more costal cartilages. The anterior mediastinum includes a quantity of loose connective tissue (such as areolar tissue), some lymph vessels, lymph glands, substernal musculature (e.g., transverse thoracic muscle), branches of the internal thoracic artery, and the internal thoracic vein. In one example, distal portion 30 of lead body 26 may be implanted substantially within the loose connective tissue and/or substernal musculature of the anterior mediastinum. In one example, distal portion 30 of lead body 26 may be implanted within the internal thoracic vein or internal thoracic artery.

In other examples, distal portion 30 of lead body 26 may be implanted in other extra-thoracic or intra-thoracic locations, including extravascular, extracardiac, or extra-pericardial locations, including the gap, tissue, or other anatomical features around the perimeter of and adjacent to the pericardium or other portion of the heart and not above sternum 12 or ribcage 18, intrapleural locations, intrapericardial locations, epicardial locations or other locations. As such, lead 22 may be implanted anywhere within the substernal space defined by the undersurface between sternum 12 and/or ribcage 18 and the body cavity.

Distal portion 30 may include or otherwise support (e.g., carry) one or more electrodes, such as electrodes 32A-32B (collectively, “electrodes 32”). Electrodes 32 may be configured to deliver low-voltage electrical pulses, e.g., for cardiac pacing) and/or may sense a cardiac electrical activity, e.g., depolarization and repolarization of the heart. As such, electrodes 32 may be referred to herein as pace/sense electrodes 32. Examples of electrodes 32 may include segmented electrodes, circumferential electrodes, ring electrodes, ribbon electrodes, short coil electrodes, paddle electrodes, hemispherical electrodes, directional electrodes, defibrillation electrodes, etc., and may be positioned at any position along distal portion 30.

Distal portion 30 may also include or otherwise support (e.g., carry) one or more voltages configured to deliver higher voltage signals, e.g., defibrillation or cardioversion shocks, such as electrodes 40A and 40B (hereinafter, “defibrillation electrodes 40”). Defibrillation electrodes 40 may be a disposed around or within the lead body 26 of the distal portion 30. In one configuration, the defibrillation electrodes 40 may each be coil electrodes formed by a conductor. The conductor may be formed of one or more conductive polymers, ceramics, metal-polymer composites, semiconductors, metals or metal alloys, including but not limited to, one of or a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole and other polymers. In another configuration, each of the defibrillation electrodes 40 may be a flat ribbon electrode, a paddle electrode, a braided or woven electrode, a mesh electrode, a directional electrode, a patch electrode or another type of electrode configured to deliver a cardioversion/defibrillation shock to the patient's heart. Defibrillation electrodes 40 may be electrically connected to one or more conductors, which may be disposed in the body wall of the lead body 26 or may alternatively be disposed in one or more insulated lumens (not shown) defined by the lead body 26. Defibrillation electrodes 40 may be connected to a common conductor such that a voltage may be applied simultaneously to both or attached to separate conductors such that each defibrillation electrode 40 may apply a voltage independent of the other defibrillation electrode.

Proximal portion 28 of lead body 26 may include one or more connectors to electrically couple lead 22 to stimulation device 14. In some examples, each of the electrodes 32 and 40 on distal portion 30 is electrically connected to a corresponding contact on the connector on proximal portion 28 via one or more electrical conductors. The connector may, for example, comprise a standard connector, such as a DF-4, IS4, EV-4, DF-1, IS-1 connector or a proprietary connector.

Stimulation device 14 may include a housing that forms a hermetic seal that protects components of stimulation device 14. The housing of stimulation device 14 may be formed of a conductive material, such as titanium or titanium alloy, which may function as a housing electrode for a particular therapy vector between the housing and distal portion 30. The stimulation device 14 may also include a connector assembly that includes electrical feedthroughs through which electrical connections are made between the one or more connectors of lead 22 and electronic components included within the housing. The housing may contain circuitry, such as processing circuitry, memory circuitry, telemetry circuitry, sensing circuitry, therapy circuitry (which may include, for example, a pulse generator(s), transformer(s), capacitor(s), or the like), switching circuitry, power circuitry (capacitors and batteries), etc.

Stimulation device 14 may generate and deliver electrical stimulation therapy, including traditional low voltage stimulation therapies (e.g., anti-tachycardia pacing, post-shock pacing, bradycardia pacing, cardiac resynchronization pacing, pacing used in conjunction with VF induction, neurostimulation pacing, etc.) as well as (optionally) traditional high voltage stimulation therapies (e.g., cardioversion or defibrillation shocks) via various electrode combinations or vectors.

Stimulation device 14 may detect a ventricular tachyarrhythmia (e.g., VT or VF) based on signals sensed using electrodes 32 and/or other electrodes described herein, such as defibrillation electrodes 40. In response to detecting the tachyarrhythmia, stimulation device 14 may generate low voltage and/or high voltage electrical stimulation therapy and deliver the electrical stimulation therapy via combinations of electrodes 32 and/or 40. Additionally or alternatively, stimulation device 14 may deliver pacing (e.g., ATP or post-shock pacing). If high voltage therapy is necessary, stimulation device 14 may deliver a cardioversion/defibrillation shock (or multiple shocks) using defibrillation electrodes 40 and/or the housing of stimulation device 14. Stimulation device 14 may generate and deliver the pacing pulses to provide anti-tachycardia pacing (ATP), bradycardia pacing, post shock pacing, pause prevention pacing or other pacing therapies or combination of pacing therapies.

As described above, patient 16 may experience sensation during pacing because of, for example, stimulation of skeletal muscles and intercostal nerves (and/or any other muscle tissue and nerve tissue) proximate electrodes 32 and/or 40 of lead 22. In accordance with techniques of this disclosure, stimulation device 14 may determine a likelihood of sensation at a target site from a pacing therapy based on a set of stimulation parameters and a set of evoked response parameters. This information may facilitate implantation of lead 22 that avoids or at least reduces undesirable sensation experienced by patient 16 patient during treatment and/or facilitate stimulation parameters settings to reduce the likelihood or the amount of sensation experienced by patient 16, thus improving patient outcomes.

Stimulation device 14 may deliver, via electrodes 32 and/or 40 of lead 22 positioned proximate a target site (e.g., a prospective implantation site), a set of stimulation pulses based on a set of stimulation parameters. Example stimulation parameters may include at least one of polarity, pulse width, pulse frequency, inter-phase delay, inter-pulse delay, stimulation amplitude (e.g., stimulation current amplitude, stimulation voltage amplitude, etc.), or stimulation vector (e.g., the two or more electrodes used to deliver stimulation and their polarities). Additionally, these parameters may be time-varying in order to, for example, ramp the amplitude during the delivery of sequential pulses in a train. In some examples, stimulation device 14 may be configured to coordinate delivery (e.g., gating) of stimulation pulses with the cardiac cycle to reduce a risk of stimulating the heart tissue (e.g., at higher pulse frequencies). In some examples, the stimulation pulses may be asynchronously delivered with the heart rate, or delivered synchronously during the refractory period (mitigating the potential of pacing during the vulnerable period which can be pro-arrhythmogenic). A sequence of pulses delivered during the refractory period may include either a single or multiple pulses.

The set of stimulation pulses to the target site may elicit a set of evoked response signals (e.g., an electrical potential generated by stimulated muscle or nervous tissue of patient 16 following presentation of a stimulus) from the target site. In general, the set of evoked response signals may be distinct from spontaneous electrical potentials generated by the nervous system of patient 16. Each evoked response signal of the set of evoked response signals may be in response to a corresponding stimulation pulse from the set of stimulation pulses delivered by electrodes 32. Electrodes 32 may sense or otherwise measure the set of evoked response signals.

Stimulation device 14 may measure a set of evoked response parameters for the set of evoked response signals. Example evoked response parameters may include at least one of latency, morphology, frequency spectra, evoked response amplitude (e.g., evoked response voltage amplitude), or sensing vector (e.g., the set of electrodes 32 measuring the evoked response). In general, the evoked response parameters may be influenced by the pacing electrodes and/or the sensing electrodes. Changing the pacing and/or sensing vectors may allow the identification of differences in proximity to target tissues, tissue anisotropy, and/or propagation direction of the evoked response.

The set of evoked response parameters may indicate a likelihood of sensation by patient 16 in response to pacing therapy (e.g., pacing therapy using the same set of stimulation parameters that elicited the set of evoked response signals). In general, one or more of the set of evoked response parameters may depend on (e.g., be related to, be a function of, etc.) the stimulation parameters of the set of stimulation pulses that elicited the evoked response signal.

Analysis of the set of evoked response parameters may indicate whether a set of stimulation parameters may result in undesirable sensation by patient 16. In some examples, the set of evoked response parameters may facilitate tissue classification. For example, a relatively small latency value may be most closely associated with nerve tissue (as opposed to any other tissue type). In another example, a relatively sharp morphology may be most closely associated with nerve tissue (as opposed to any other tissue type). In yet another example, a relatively round morphology may be most closely associated with muscle tissue (as opposed to any other tissue type). Stimulation of muscle tissue and nerve tissue proximate electrodes 32 of lead 22 may cause patient 16 to experience undesirable sensation.

In some examples, analysis of the set of evoked response parameters by processing circuitry may include taking a single measurement at a given setting, taking and averaging multiple measurements, and/or taking multiple measurements and removing outliers. In some examples, the analysis may include automatically or manually ramping a stimulation parameter to determine a minimum threshold energy for eliciting the evoked response signal. In some examples, the analysis may include extracting characteristics of the ramp-test to evaluate stimulation plateaus or other features. In some examples, analysis may include extracting frequency or morphology characteristics of the signal and comparing to thresholds and/or template.

Regarding extracting frequency or morphology characteristics, stimulation device 14 may determine that an evoked response has a relatively sharp or round morphology based on the slope of the evoked response within the evaluation window. For example, stimulation device 14 may determine a derivative or differential signal based on the evoked response signal to determine a slope of the evoked response signal. Stimulation device 14 may then compare the differential signal to a maximum slope threshold and/or a minimum slope threshold. A maximum slope having a large positive value may indicate a rapid signal increase, and a minimum slope having a large negative value may indicate a rapid signal decrease, both of which may result in a relatively “sharp” signal morphology. In some examples, responsive to the differential signal satisfying the maximum slope threshold and/or the minimum threshold, stimulation device 14 may determine that the evoked response has a relatively sharp morphology. Responsive to the differential signal not satisfying the maximum slope threshold and/or the minimum threshold, stimulation device 14 may determine that the evoked response has a relatively “round” morphology.

Analysis of the set of evoked response parameters may help guide implantation of lead 22 and/or configuration of therapeutic stimulation delivered via lead 22 to avoid undesirable sensation. For example, if stimulation device 14 determines, based on the set of stimulation parameters and the set of evoked response parameters, a likelihood of sensation at the target site from a pacing therapy, stimulation device 14 may output (e.g., for display) the determination. In cases in which the stimulation device 14 is implanted, for example, stimulation device 14 may transmit the determination to external device 20 for display to a physician. The output may include, for example, a level of risk (e.g., low, medium, high, etc.) of sensation, one or more of the set of evoked response parameters, one or more of the stimulation parameters of the set of stimulation parameters, etc. In some examples, stimulation device 14 may be configured, e.g., based on commands from external device 20, to iteratively test combinations of stimulation and sensing parameters. External device 20 may present the likelihoods of sensation associated with each combination to a physician and, in some examples, recommend options for the physician to consider based on the determined likelihood of sensation. A physician may reposition lead 22 based on the output from stimulation device 14. In some examples, the physician may reposition lead 22 until stimulation device 14 determines that there is no risk (or an acceptable level of risk) of sensation at the target site from the pacing therapy.

Although stimulation device 14 is primarily described herein as determining and analyzing the set of evoked response parameters, it should be understood that any computing device of system 10 may perform such determination and/or analysis. For example, external device 20 may obtain the sensed evoked response signal and determine the evoked response parameters for analysis or receive the evoked response parameters and perform the analysis to determine, based on the set of stimulation parameters and the set of evoked response parameters, a likelihood of sensation at the target site from a pacing therapy.

As such, stimulation device 14 may be in wireless communication with external device 20 (e.g., a computing device for use by a patient, a clinician, etc.) to transmit information to external device 20, be programmed by external device 20, or otherwise communicate with external device 20.

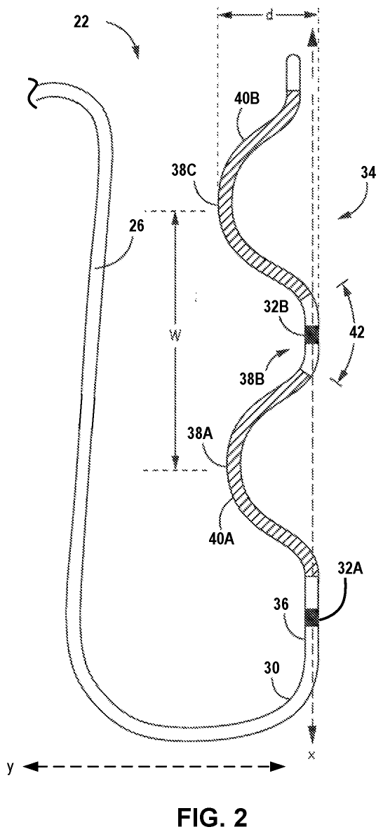

FIG. 2 is conceptual diagram of lead 22. As shown in FIG. 2, distal portion 30 may define an undulating configuration 34 distal to a substantially linear portion 36 (“linear portion 36”). In particular, distal portion 30 may define an undulating pattern, e.g., (zig-zag, meandering, sinusoidal, serpentine, or other pattern) as it extends toward the distal end of distal portion 30. Undulating configuration 34 may be substantially disposed in a plane defined by the longitudinal axis (“x”) and a transverse axis (“y”). In some examples, lead body 26 may not have linear portion 36 as it extends distally, but instead undulating configuration 34 may begin immediately after the bend. It will be appreciated that FIG. 2 illustrates an example lead configuration and other lead configurations may be used, including for example, straight configurations.

Undulating configuration 34 may include a plurality of peaks along the length of distal portion 30, such as peaks 38A-38C (collectively, “peaks 38”). Undulating configuration 34 may include any number of peaks 38. For example, the number of peaks 38 may be fewer or greater than three depending on the frequency of the undulation configuration 34. Undulating configuration 34 may define a peak-to-peak distance “d,” (shown in FIG. 2), which may be variable or constant along the length of undulating configuration 34. As shown in FIG. 2, undulating configuration 34 may define a substantially sinusoidal configuration, with a constant peak-to-peak distance “d” of approximately 2.0-5.0 centimeters (cm). Undulating configuration 34 may also define a peak-to-peak width “w,” (shown in FIG. 2), which may also be variable or constant along the length of undulating configuration 34. In other instances, undulating configuration 34 may define other shapes and/or patterns, e.g., S-shapes, wave shapes, or the like.

Distal portion 30 may include defibrillation electrodes, such as defibrillation electrodes 40. Defibrillation electrodes 40 may be configured to deliver a cardioversion/defibrillation shock. Defibrillation electrodes 40 may include a plurality of sections or segments spaced a distance apart from each other along the length of distal portion 30. In some examples, defibrillation electrodes 40 may be a coil electrode formed by a conductor. The conductor may be formed of one or more conductive polymers, ceramics, metal-polymer composites, semiconductors, metals or metal alloys, including but not limited to, one of or a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole and other polymers. In another configuration, defibrillation electrodes 40 may be a flat ribbon electrode, a paddle electrode, a braided or woven electrode, a mesh electrode, a directional electrode, a patch electrode or another type of electrode configured to deliver a cardioversion/defibrillation shock to the patient's heart.

Distal portion 30 may define one or more gaps 42 between adjacent defibrillation electrodes 40. Gaps 42 may define any length. One or more electrodes be disposed within respective gaps 42. For example, electrodes 32 may be disposed within respective gaps 42. Additionally or alternatively, electrodes 32 may be disposed along distal portion 30 of lead 22 (e.g., proximal to segment 40A and/or distal to segment 40B). Electrodes 32 may be examples of electrodes 32 shown in FIG. 1. Electrodes 32 and/or defibrillation electrodes 40 may be configured to deliver stimulation energy in accordance with techniques of this disclosure.

As described above, electrodes 32 may be electrically coupled to stimulation device 14 via one or more connectors. In some examples, electrodes 32 may be electrically coupled to stimulation device 14 via one connector with multiple contacts. Lead 22 may include conductors that couple to the respective contacts of the connector. In some examples, each of defibrillation electrodes 40 and electrodes 32 may be electrically connected to a corresponding connector on proximal portion 28. Defibrillation electrodes 40 may be used to provide defibrillation therapy. Any of electrodes 32 may be used for pacing with another lead electrode or the housing electrode (or a surface electrode, such as the external electrode described in greater detail below).

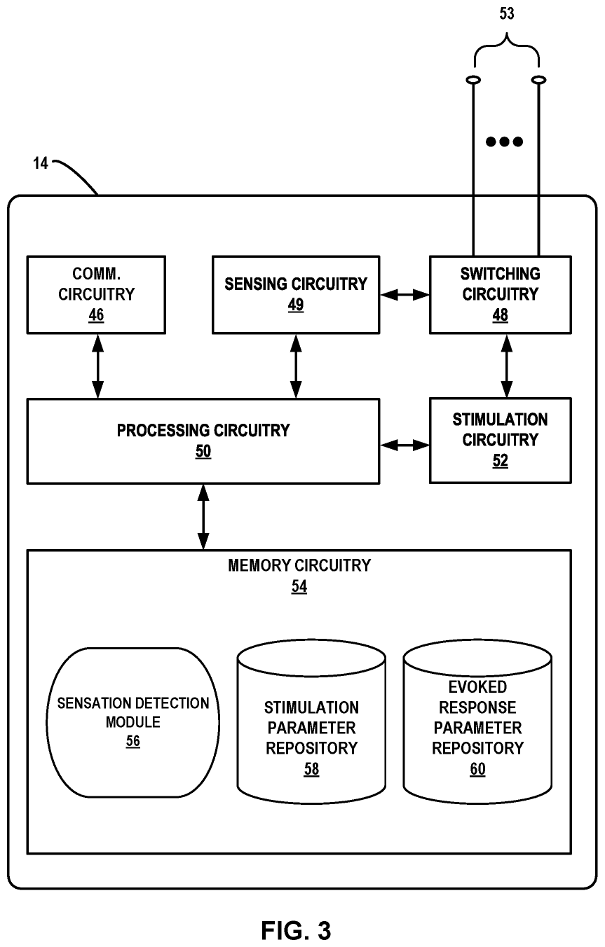

FIG. 3 is a block diagram illustrating an example configuration of stimulation device 14 in accordance with techniques of this disclosure. As shown in FIG. 3, stimulation device 14 includes communication circuitry 46 (“COMM circuitry 46”), switching circuitry 48, sensing circuitry 49, processing circuitry 50, stimulation circuitry 52, and memory circuitry 54. Stimulation device 14 may be electrically connected to electrodes 53. In examples where system 10 includes lead 22, stimulation device 14 may be electrically connected to electrodes 53 via lead 22. Electrodes 53 may be examples of electrodes 32, defibrillation electrodes 40, the housing of stimulation device 14, or any other electrode of system 10. Proximal portion 28 of lead 22 may be electrically connected to stimulation device 14.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Stimulation circuitry 52 may be configured to generate and deliver electrical therapy. Stimulation circuitry 52 may include one or more pulse generators, capacitors, and/or other components capable of generating and/or storing energy to deliver as pacing therapy, defibrillation therapy, cardioversion therapy, other therapy, or a combination of therapies. In some instances, stimulation circuitry 52 may include a first set of components configured to provide pacing therapy and a second set of components configured to provide anti-tachyarrhythmia shock therapy. In other instances, stimulation circuitry 52 may utilize the same set of components to provide both pacing and anti-tachyarrhythmia shock therapy. In still other instances, stimulation circuitry 52 may share some of the pacing and shock therapy components while using other components solely for pacing or shock delivery.

Stimulation circuitry 52 may include charging circuitry, one or more charge storage devices, such as one or more capacitors, and switching circuitry that controls when the capacitor(s) are discharged to electrodes 53 and the widths of pulses. Charging of capacitors to a programmed pulse amplitude and discharging of the capacitors for a programmed pulse width may be performed by stimulation circuitry 52 according to control signals received from processing circuitry 50, which are provided by processing circuitry 50 according to parameters stored in memory circuitry 54. Processing circuitry 50 controls stimulation circuitry 52 to deliver the generated therapy to the heart via one or more combinations of electrodes 53, e.g., according to parameters stored in memory circuitry 54. Stimulation circuitry 52 may include switch circuitry to select which of the available electrodes 53 are used to deliver the therapy, e.g., as controlled by processing circuitry 50.

Stimulation circuitry 52 may be selectively coupled to electrodes 53 via switching circuitry 48 as controlled by processing circuitry 50 to, for example, deliver a set of stimulation pulses to tissue of patient 16. Stimulation circuitry 52 may deliver the set of stimulation pulses based on a set of stimulation parameters stored in a stimulation parameter repository 58 in memory circuitry 54. The stimulation parameters stored in stimulation parameter repository may include one or more stimulation vectors and stimulation amplitudes (voltage or current).

Sensing circuitry 49 may be selectively coupled to electrodes 53 via switching circuitry 48 as controlled by processing circuitry 50 to, for example, sense electrical signals (e.g., evoked response signals) from tissue of patient 16. Sensing circuitry 49 may sense the electrical signals based on a set of sensing parameters stored in memory circuitry 54. In other words, the set of sensing parameters may configure sensing circuitry 49 for sensing evoked responses. In some examples, sensing circuitry 49 may include one or more filters and amplifiers for filtering and amplifying signals received from electrodes 32. Sensing circuitry 49 may include analog-to-digital conversion circuitry for converting the signals to digital samples for analysis by processing circuitry 50 and/or storage in memory circuitry 54. Processing circuitry 50 may analyze the sensed evoked response signals to determine evoked response parameters. Processing circuitry 50 may then store the evoked response parameters in an evoked response parameter repository 60 in memory circuitry 54.

COMM circuitry 46 may include any suitable hardware, firmware, software or any combination thereof for communicating with another device, such as external device 20, another networked computing device, or another IMD or sensor. Under the control of processing circuitry 50, COMM circuitry 46 may receive downlink telemetry from, as well as send uplink telemetry to external device 20 or another device with the aid of an internal or external antenna. COMM circuitry 46 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, memory circuitry 54 includes computer-readable instructions that, when executed by processing circuitry 50, cause processing circuitry 50, and in turn stimulation device 14, to perform various functions attributed to stimulation device 14 herein. Memory circuitry 54 may include any volatile, non-volatile, magnetic, optical, or electrical media, such as a random-access memory (RAM), read-only memory (ROM), non-volatile RAM (NVRAM), electrically-erasable programmable ROM (EEPROM), ferroelectric RAM (FRAM), dynamic random-access memory (DRAM), flash memory, or any other digital media. Memory circuitry 54 may store, as examples, programmed values for one or more operational parameters of processing circuitry 50. Memory circuitry 54 may also store data collected by stimulation device 14 for transmission to another device using COMM circuitry 46 and/or further analysis by processing circuitry 50.

Memory circuitry 54 may store a sensation detection module 56 executable by processing circuitry 50. When executing sensation detection module 56, processing circuitry 50 may determine a likelihood of sensation at the target site from a pacing therapy based on the set of stimulation parameters in stimulation parameter repository 58 and the set of evoked response parameters in evoked response parameter repository 60. In some examples, sensation detection module 56 may configure processing circuitry 50 to determine the likelihood of sensation by determining whether at least one evoked response parameter of the set of evoked response parameters satisfies a corresponding evoked response parameter condition. In some examples, an evoked response parameter may satisfy a corresponding evoked response parameter condition when the evoked response parameter is equal to or greater than a corresponding evoked response parameter threshold.

For example, responsive to a set of stimulation pulses having a stimulation current amplitude of 10 milliamps (mA), tissue at a target site may produce a set of evoked response signals having an evoked response voltage amplitude of about 75 microvolts (μV). The corresponding evoked response parameter threshold for evoked response voltage amplitude may be 10 μV. Sensation detection module 56 may determine that the evoked response voltage amplitude parameter satisfies the corresponding evoked response parameter condition because 75 82 V is greater than 10 μV. Thus, processing circuitry 50 may use sensation detection module 56 to determine that there is a likelihood of sensation at the target site from a pacing therapy.

The threshold evoked response voltage of 10 μV is merely an example, and other threshold evoked response voltages, e.g., within a range from 10-100 μV, may be used in accordance with the techniques of this disclosure. In general, the threshold evoked response voltage may be greater than a noise floor of the signal sensed subsequent to delivering the stimulation pulse.

In some examples, sensation detection module 56 may include a machine learning module (not shown). In such examples, processing circuitry 50 may apply one or more machine learning models to determine a likelihood of sensation at the target site from a pacing therapy based on the set of stimulation parameters in stimulation parameter repository 58 and the set of evoked response parameters in evoked response parameter repository 60. In some examples, the machine learning models may be trained by optimizing an objective function. The objective function may represent a loss function that compares (e.g., determines a difference between) output data generated by the model from the training data and labels (e.g., ground-truth labels) associated with the training data. For example, the loss function may evaluate a sum or mean of squared differences between the output data and the labels. In some examples, the labels may derive from patient input regarding the presence or absence of sensation at the target site from a pacing therapy.

Patient input may be obtained perioperatively, postoperatively, etc.

In some examples, the machine learning models may be trained using supervised learning techniques. For example, the machine learning models may be trained on a training dataset that includes training examples of user inputs labeled as belonging to the “sensation” class or “no sensation” class. In general, example machine learning techniques that may be employed to generate one or more machine learning models may include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In general, sensation detection module 56 may determine that a set of evoked response signals having parameter values substantially deviating (e.g., the deviation is not likely due to noise) from baseline values or programmed threshold values may indicate a likelihood of sensation at the target site from a pacing therapy. Thus, a set of evoked response signals in itself (as opposed to no response to a set of stimulation pulses) may indicate a likelihood of sensation at the target site from a pacing therapy, and analysis of the deviations of the evoked response parameters in view of the stimulation parameters may indicate the degree (e.g., high, medium, low, etc.) of likelihood of sensation at the target site from a pacing therapy.

Stimulation device 14 may output (e.g., transmission via COMM circuitry 46 to external device 20 for display) the determination by stimulation detection module 56. The output may include, for example, a positive or negative indication of extracardiac stimulation, a level of risk (e.g., low, medium, high, etc.) of sensation, one or more of the set of evoked response parameters, one or more of the stimulation parameters of the set of stimulation parameters, one or more of the conditions satisfied by the set of evoked response parameters, etc.

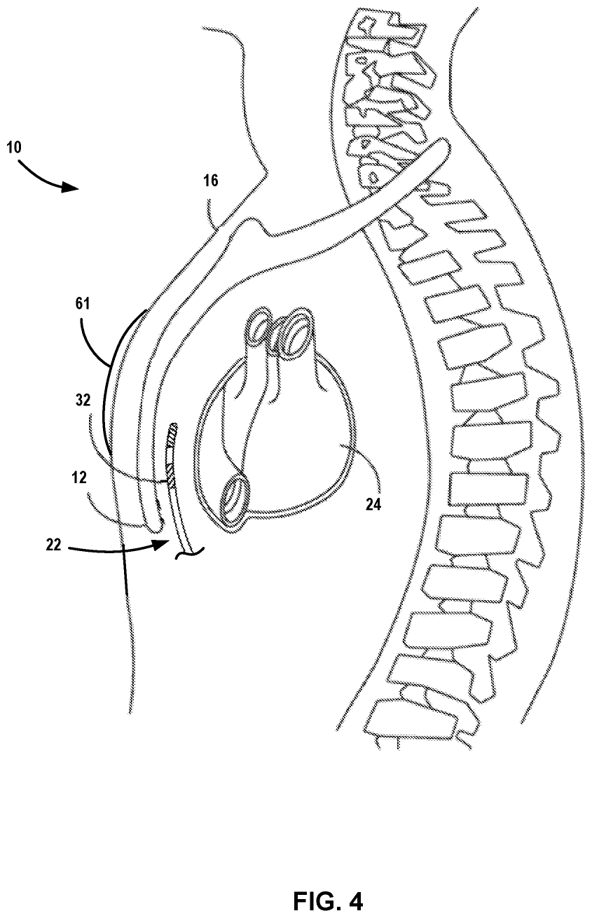

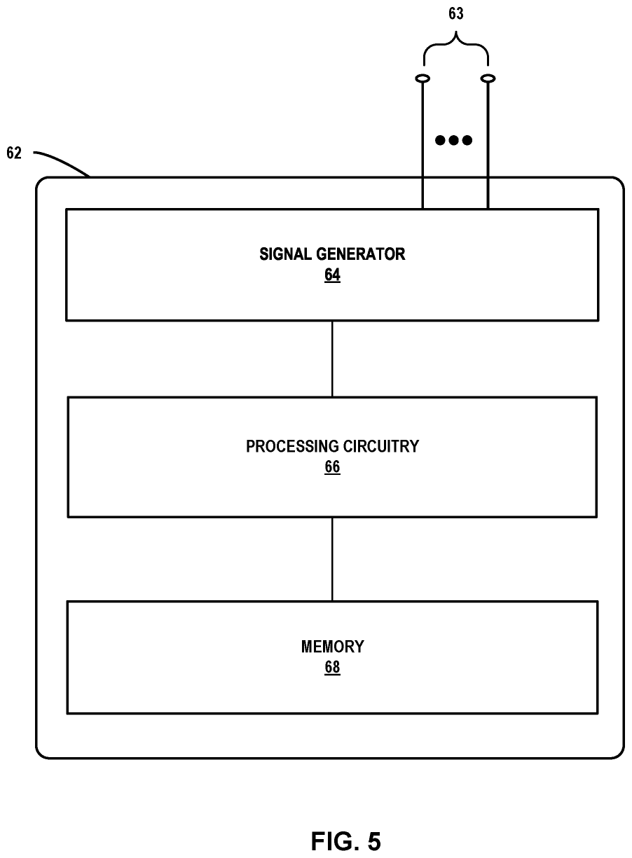

FIG. 4 is a conceptual diagram of system 10 further including an external electrode 61. System may include an external electroporation device coupled to lead 22 (or other extravascular elongated structure) and external electrode 61. The external electroporation device may include a signal generator (see FIG. 5 below). External electrode 61 may be wearable by, e.g., attached to, patient 16. System 10 may perform IRE to tissue responsible for undesirable sensation in accordance with techniques of this disclosure using electrodes 32 of lead 22 and external electrode 61. In some examples, external electrode 61 may a removable pad or patch configured to be placed on the patient's body. External electrode 61 may be placed and replaced to facilitate irreversibly electroporating various target tissues (e.g., various sites proximate to sternum 12) for ablation.

FIG. 5 is an example block diagram of a device configured to incapacitate tissue. In some examples, the device may be an ablation device, such as an electroporation device 62. System 10 may include electroporation device 62. Electroporation device 62 may deliver electroporation energy to tissue responsible for sensation during pacing therapy to reduce or eliminate sensation. For instance, delivering irreversible electroporation (IRE) energy to the tissue may physiologically modify the cells of the tissue to which the energy is applied. In some examples, depending on the characteristics of the electrical pulses, the electroporated cells may be irreversibly electroporated such that sensation during pacing is reduced or eliminated entirely.

As used herein, electroporation refers to a phenomenon that causes cell membranes to become “leaky” (that is, permeable for molecules for which the cell membrane may otherwise be impermeable or semipermeable). Electroporation, which may also be referred to as electropermeabilization, pulsed electric field treatment, non-thermal irreversible electroporation, irreversible electroporation, high frequency irreversible electroporation, nanosecond electroporation, or nanoelectroporation, may involve the application of high-amplitude pulses to cause physiological modification (i.e., permeabilization) of the cells of the tissue to which the energy is applied. These pulses may be short (e.g., nanosecond, microsecond, or millisecond pulse width, such as about 100 nanoseconds to about 20 milliseconds) in order to allow the application of high voltage (e.g., about 100 to 5000 volts), high current (e.g., 20 or more amps) without long duration(s) of electrical current flow that may otherwise cause significant tissue heating and muscle stimulation. In some examples, the number of pulses per second may be from about 1 to about 500. The pulsed electric energy may induce the formation of microscopic defects that result in hyperpermeabilization of the cell membrane. Depending on the characteristics of the electrical pulses, an electroporated cell can survive electroporation, referred to as “reversible electroporation,” or die, referred to as IRE. Reversible electroporation may be used to transfer agents, including genetic material and other large or small molecules, into targeted cells for various purposes, including the alteration of the action potentials of cardiac myocytes. In general, IRE may be an acute procedure, meaning it may only need to be performed once to achieve the advantages disclosed herein. IRE may be performed at any time (e.g., perioperatively, postoperatively, etc.). However, IRE is primarily described herein as being performed perioperatively.

As shown in FIG. 5, electroporation device 62 may include a signal generator 64, processing circuitry 66 (which may be substantially similar to processing circuitry 50 of FIG. 3), and memory circuitry 68 (which may be substantially similar to memory circuitry 54 of FIG. 3). Electroporation device 62 and stimulation device 14 may be integrated into the same device. For example, stimulation device 14 and electroporation device 62 may be contained in the same housing. In some examples, stimulation device 14 may include electroporation device 62 or vice versa. In other examples, stimulation device 14 and electroporation device 62 may be distinct devices (e.g., stimulation device 14 and electroporation device 62 do not share the same housing).

Signal generator 64 may be selectively coupled to electrodes 63. Electrodes 63 may be electrodes 32 or other electrodes described above. Signal generator 64 may be configured to provide electrical pulses to electrodes 63 to perform an electroporation procedure. For instance, signal generator 64 may be configured and programmed to deliver pulsed, high-voltage electric fields appropriate for achieving desired pulsed, high-voltage ablation via IRE and/or pulsed RF ablation. The pulsed-field energy may be sufficient to induce cell death for purposes of destroying the ability of the so-ablated tissue to propagate or conduct electrical signals associated with undesirable sensation. In this way, electroporation device 62, via signal generator 64, may deliver electroporation energy to tissue responsible for sensation during pacing, thereby irreversibly electroporating the tissue.

Electroporation device 62 may be electrically connected to lead 22 (or any other extracardiac elongated structure in accordance with techniques of this disclosure). Lead 22 may be navigated to a target site within patient 16 such that electrodes 63 are proximate to the target site. When proximate to the target site, electrodes 63 may be oriented relative to heart 24 of patient 16. For instance, in examples where electrodes 63 are segmented electrodes (e.g., directional electrodes), electrodes 63 may be oriented toward a posterior sternal surface such that the electrical fields produced by electrodes 63 are simultaneously directed toward target tissue for ablation and away from heart 24.

Signal generator 64 may provide electrical pulses to perform an electroporation procedure to extracardiac tissue within the extra-thoracic space, or other tissues within the body, such as renal tissue, or airway tissue. Electroporation utilizes high amplitude pulses to effectuate a physiological modification (i.e., permeabilization) of the cells to which the energy is applied. Such pulses may preferably be short (e.g., nanosecond, microsecond, or millisecond pulse width) in order to allow application of high voltage, high current (for example, 20 or more amps) without long duration of electrical current flow that results in significant tissue heating. In particular, the pulsed energy induces the formation of microscopic pores or openings in the cell membrane.

Signal generator 64 may be configured and programmed to deliver pulsed, high voltage electric fields appropriate for achieving desired pulsed, high voltage ablation (or pulsed field ablation). As a point of reference, the pulsed, high voltage, non-radiofrequency, ablation effects of the present disclosure may be distinguishable from DC current ablation, as well as thermally-induced ablation attendant with conventional RF techniques. For example, the pulse trains delivered by signal generator 64 may be delivered at a frequency less than 3kHz, and in an exemplary configuration, 1kHz, which is a lower frequency than radiofrequency treatments. The pulsed-field energy in accordance with the present disclosure may be sufficient to induce cell death for purposes of preventing sensory response to cardiac pacing or other electrical stimulation as described herein.

In some examples, electrodes 32 may deliver therapeutic biphasic pulses having a preprogrammed pattern and duty cycle. For example, each pulse cycle may include an applied voltage amplitude A, a pulse width B (in microseconds (μs)), an inter-phase delay C (in μs), an inter-pulse delay D (in μs), and a pulse cycle length E. In an exemplary configuration, the pulse width B may be 1-15 μs, the inter-phase delay C may be 0-4 μs, the inter-pulse delay D may be 5-30,000 μs, the pulse train may include 20-1000 pulses, and the applied voltage may be approximately 300-4000 V. In some examples, the pulse width may be set to 5 μs, the inter-phase delay may be 5us, the inter-pulse delay may be 800 μs, and the pulse train may include 80 pulses with an applied voltage of 700V.

Such a pulse train when delivered from a bipolar electrode array may produce lesions in tissue in the range of approximately 2-3mm deep. Increased voltage may correspondingly increase the lesion depth. In another example, four pulse trains may be delivered at each target tissue site.

The pulsed field of energy may be delivered in a bipolar fashion, in monophasic or biphasic pulses. The application of biphasic electrical pulses may produce unexpectedly beneficial results in the context of tissue ablation. With biphasic electroporation pulses, the direction of the pulses completing one cycle alternates in a few microseconds. As a result, the cells to which the biphasic electrical pulses are applied may undergo alternation of electrical field bias. Changing the direction of bias reduces prolonged post-ablation depolarization and/or ion charging. As a result, prolonged muscle excitation may be reduced. Further, biphasic electrical pulses may overcome the high impedance characteristics of fatty cells that are often problematic in ablation procedures.

In some examples, the pulse width B may be 5 μs or less, based at least in part on the evaluation of bubble output at high voltages and/or evidence of thermal effects on the tissue surface. As for the presence of bubbles, a pulse width of greater than 15 μs may be more likely to produce significant gas bubble volume and pulse widths of 20 μs or longer may produce thermal effects on the tissue surface. No loss of efficacy has been observed when going from 100 μs to 5 μs pulse width. Further, pulses with a pulse width as short as 5 μs may reduce non-collateral tissue stimulation.

An applied voltage amplitude of between approximately 200V and approximately 300V may be the threshold amplitude at which irreversible damage is caused to cells that are in direct contact with the electrodes 32. In general, irreversible electroporative effects may be obtained if the E-field distribution is oriented such that the highest field strength is applied along (or parallel to) the long axis of the targeted cells. However, maximal irreversible electroporative effects may be achieved if multiple field vectors are applied to the targeted cells because different cells may react differently to a particular E-field orientation. The polarity of adjacent electrodes 32 may be alternated to achieve the widest variety of field directions possible. If more than one vector is used, a larger percentage of cells may be affected and a more complete lesion may be created. Although not shown, additional distal portion 30 configurations may be used to produce a variety of E-field vectors. As a non-limiting example, the distal portion 30 may include a mesh-covered balloon, a balloon with embedded surface electrodes, or a splined basket with multiple electrodes. Additionally or alternatively, additional electrodes may be added to existing devices to deliver some of the pulses to add a new field direction.

Electroporation device 62 may deliver electroporation energy to a target site to prevent or reduce undesirable sensation at the target site. For example, as described above, stimulation device 14 may determine a likelihood of sensation at a target site from a pacing therapy based on a set of stimulation parameters and a set of evoked response parameters. If there is a likelihood of sensation at the target site from the pacing therapy, electroporation device 64 may deliver electroporation energy to irreversibly electroporate the target site. Following delivery of the electroporation energy, stimulation device 14 may re-determine the likelihood of sensation at the post-ablated target site from a pacing therapy based on a set of stimulation parameters and a set of evoked response parameters. If IRE is successful, the set of evoked response parameters should be such that stimulation device 14 may determine that there is no risk (or an acceptable risk) of sensation at the post-ablated target site from a pacing therapy. That is, the set of evoked response parameters of the set of evoked response signals post-IRE should be different from the set of evoked response parameters of the set of evoked response signals pre-IRE such that stimulation device 14 reaches a different determination regarding the likelihood of sensation post-IRE.

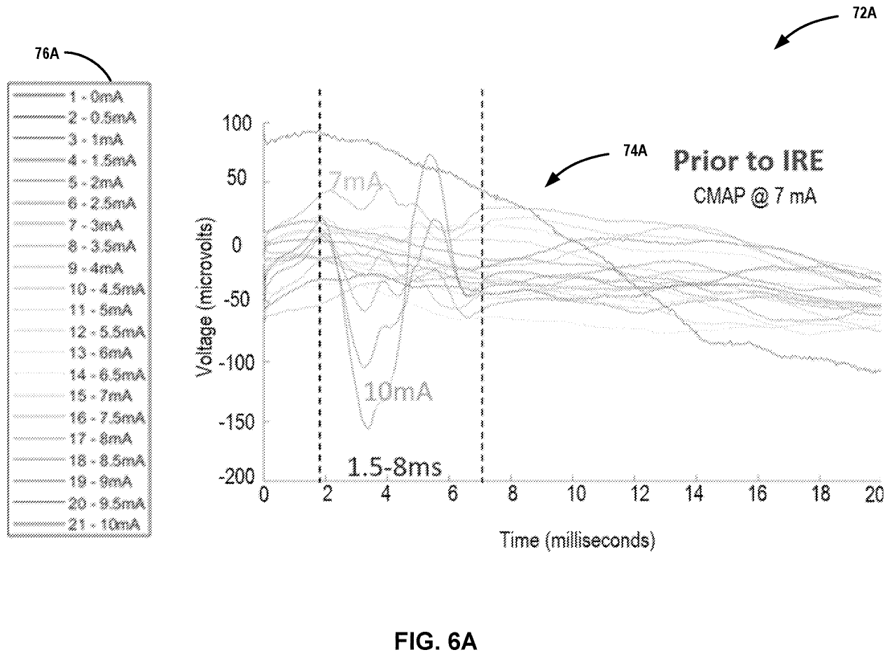

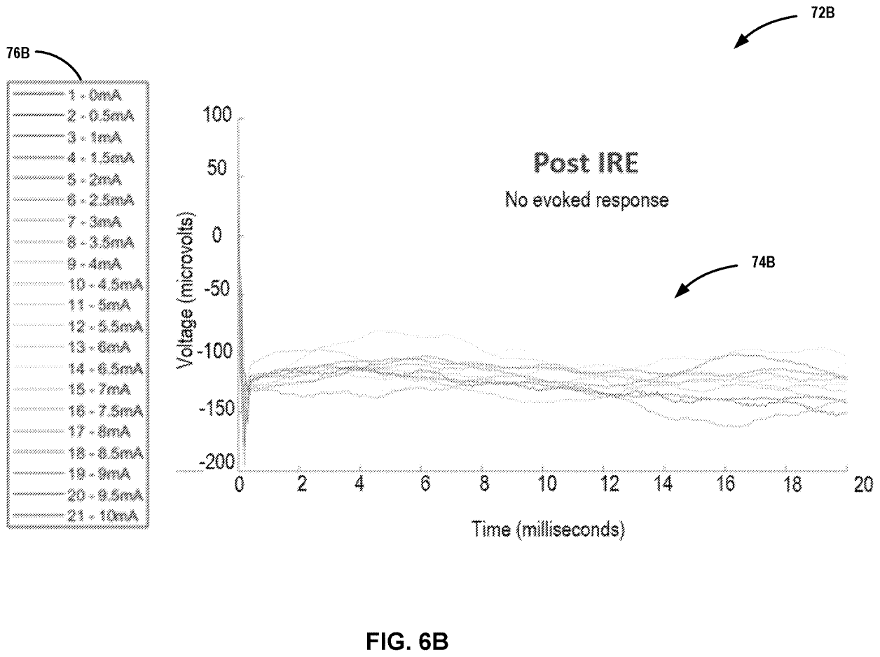

FIG. 6A is a chart 72A illustrating an example set of evoked response signals 74A produced by tissue in response to different stimulation amplitudes, prior to an IRE procedure. In the example of FIG. 6A, each stimulation pulse of a set of stimulation pulses delivered to a target site has a corresponding stimulation current amplitude ranging from 0 mA to 10 mA, as indicated by legend 76A. Each evoked response signal of set of evoked response signals 74A is in response to a corresponding stimulation pulse from the set of stimulation pulses.

As shown in FIG. 6A, evoked response parameters of an evoked response signal may depend on the extent to which tissue is captured by a stimulation pulse, which may in turn depend on stimulation parameters of the corresponding stimulation pulse. For example, stimulation pulses with a relatively small stimulation pulse current amplitude (e.g., 1 mA) may elicit an evoked response signal having no distinct morphological features (e.g., absence of appreciable peaks and valleys) and a relatively small evoked response amplitude (e.g., substantially 0 voltage). Accordingly, sensation detection module 56 may determine there is not a likelihood of sensation at a target site from a pacing therapy having a stimulation pulse current amplitude of about 1 mA pre-IRE.

Conversely, stimulation pulses with a relatively large stimulation pulse current amplitude (e.g., 10 mA) may elicit an evoked response signal having distinct morphological features (e.g., presence of appreciable peaks and valleys) and a relatively large evoked response amplitude (e.g., a 200 μV amplitude having a range from a minimum voltage of −150 μV to a maximum voltage of 50 μV). Accordingly, sensation detection module 56 may determine there is a likelihood of sensation at a target site from a pacing therapy having a stimulation pulse current amplitude of about 10 mA pre-IRE.

FIG. 6B is a chart 72B illustrating an example set of evoked response signals 74B produced by tissue following an IRE procedure in accordance with techniques of this disclosure. In the example of FIG. 6B, each stimulation pulse of a set of stimulation pulses delivered to a target site has a corresponding stimulation current amplitude ranging from 0 mA to 10 mA, as indicated by legend 76B. Each evoked response signal of set of evoked response signals 74B is in response to a corresponding stimulation pulse from the set of stimulation pulses.

As shown in FIG. 6B, a set of stimulation pulses may not elicit a set of evoked response signals (and in turn indicating a lower likelihood of sensation) from a post-ablated target site. This difference may be particularly clear when comparing set of evoked response signals 74A of chart 72A and comparing set of evoked response signals 74B of chart 72B. As shown in FIG. 6B, following ablation of the target site, even stimulation pulses with a relatively large stimulation pulse current amplitude (e.g., 10 mA) may only elicit an evoked response signal having no distinct morphological features (e.g., absence of appreciable peaks and valleys) and a small evoked response amplitude (e.g., substantially 0 voltage). Accordingly, sensation detection module 56 may determine there is not a likelihood of sensation at a target site from a pacing therapy having a stimulation pulse current amplitude of about 10 mA post-IRE.

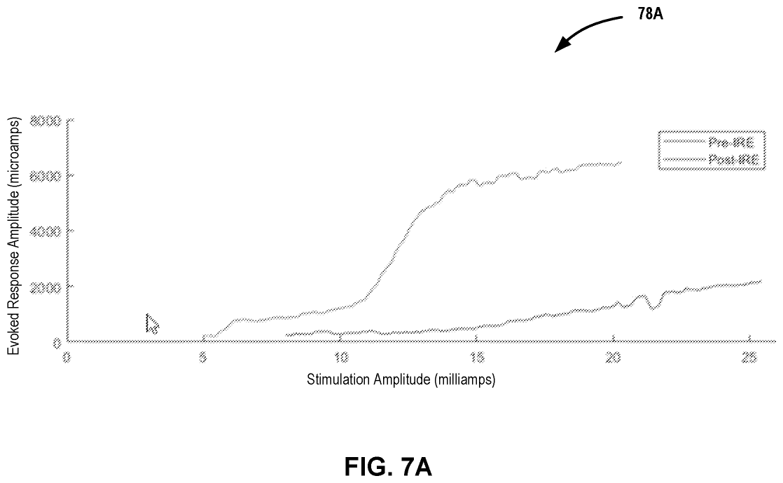

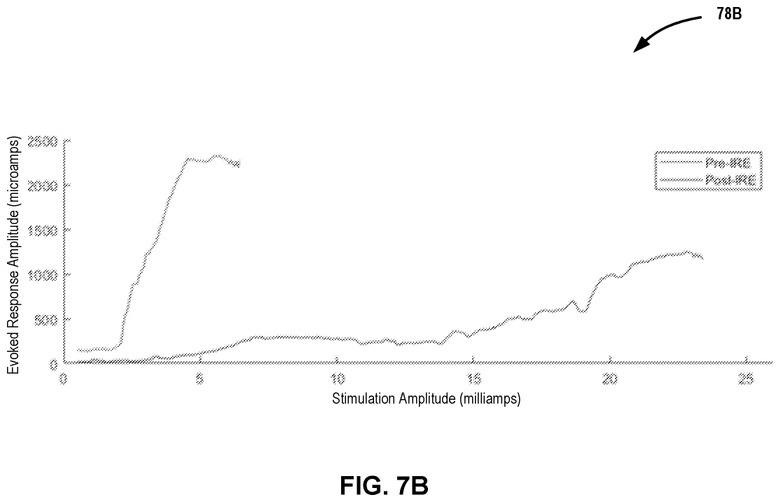

FIGS. 7A and 7B are charts 78A and 78B, respectively, illustrating example sets of evoked response signals produced by tissue prior to and following an irreversible electroporation procedure in accordance with techniques of this disclosure. As shown in FIGS. 7A and 7B, following ablation of a target site, one or more evoked response parameters (e.g., evoked response amplitude) of the set of evoked response signals produced by the tissue at the target site are reduced, potentially indicating a reduced likelihood of sensation at the target site from a pacing therapy.

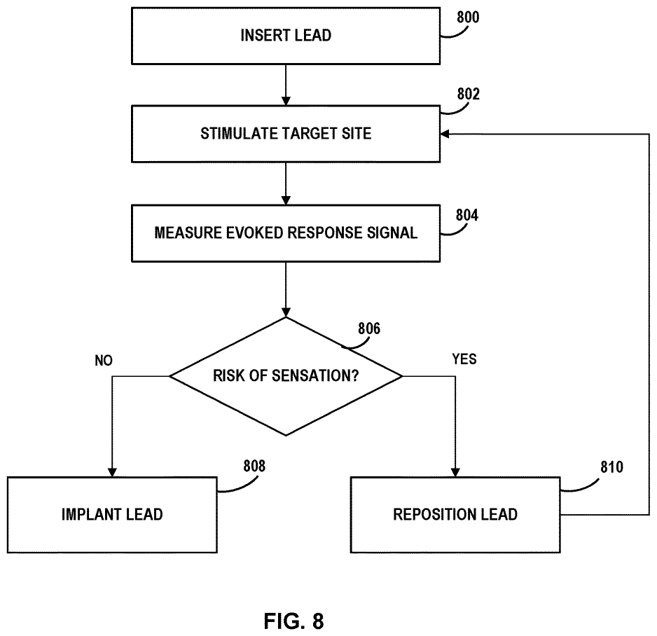

FIG. 8 is a flow diagram of an example technique for using system 10 in accordance with techniques of this disclosure. Lead 22 may be inserted into the body of patient 16 (800). Lead 22 may be navigated to a target site (e.g., a location proximate heart 24) within patient 16 such that electrodes 32 are proximate to the target site.

Lead 22 may deliver a set of stimulation pulses to the target site (802). For instance, lead 22 may be electrically coupled to stimulation device 14. Stimulation circuitry 52 of stimulation device 14 may provide electrical pulses to electrodes 32 based on a set of stimulation parameters stored in stimulation parameter repository 58.

Electrodes 32 may measure a set of evoked response signals from the target site that is produced in response to the set of stimulation pulses (804). Processing circuitry 50 may determine a set of evoked response parameters based on the sensed set of evoked response signals and store the set of evoked response parameters in evoked response parameter repository 60.

When executing sensation detection module 56, processing circuitry 50 may determine a likelihood of sensation at the target site from a pacing therapy based on the set of stimulation parameters in stimulation parameter repository 58 and the set of evoked response parameters in evoked response parameter repository 60 (806). In some examples, processing circuitry 50 may be configured to determine the likelihood of sensation by determining whether at least one evoked response parameter of the set of evoked response parameters satisfies a corresponding evoked response parameter condition. In some examples, sensation detection module 56 may include a machine learning module that applies one or more machine learning models to determine a likelihood of sensation at the target site from a pacing therapy based on the set of stimulation parameters in stimulation parameter repository 58 and the set of evoked response parameters in evoked response parameter repository 60. In some examples, processing circuitry 50 may transmit the likelihood of sensation (e.g., to a physician), display the likelihood of sensation, select pacing stimulation parameters based on the set of evoked response parameters, etc.

In any case, responsive to processing circuitry 50 determining that there is no likelihood of sensation (or an acceptable level of risk) at the target site from the pacing therapy (“NO” branch of 806), lead 22 may be implanted at the target site (808).

Responsive to sensation detection module 56 determining that there is a likelihood of sensation at the target site from the pacing therapy (“YES” branch of 806), lead 22 may be repositioned (e.g., at another target site) (810), and stimulation circuitry 52 of stimulation device 14 may provide a set of stimulation pulses to the target site as described above (802).

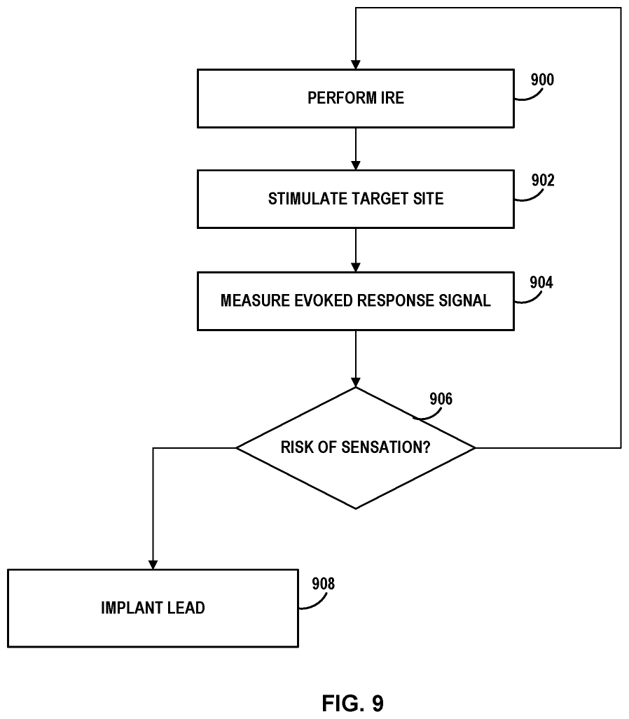

FIG. 9 is a flow diagram of an example technique for using system 10 in accordance with techniques of this disclosure. Electrodes 32 of lead 22 may be proximate to a target site. If there is a likelihood of sensation at the target site, electroporation device 62 may deliver electroporation energy via lead 22 to irreversibly electroporate tissue at the target site responsible for undesirable sensation (900). In some examples, whether there is a likelihood of sensation may be determined as described with respect to FIG. 8. For example, lead 22 may be inserted into the body of patient 16 and deliver a set of stimulation pulses to a target site. Electrodes 32 may measure a set of evoked response signals from the target site that is produced in response to the set of stimulation pulses. When executing sensation detection module 56, processing circuitry 50 may determine a likelihood of sensation at the target site from a pacing therapy based on the set of stimulation parameters and a set of evoked response parameters determined from the set of evoked response signals.

Electroporation device 62 may include signal generator 64 that provides electrical pulses to electrodes 32 to perform an electroporation procedure. For instance, signal generator 64 may deliver pulsed, high-voltage electric fields appropriate for achieving desired pulsed, high-voltage ablation via IRE and/or pulsed RF ablation.

The pulsed-field energy in accordance with this disclosure may be sufficient to induce cell death for purposes of destroying the ability of the so-ablated tissue to propagate or conduct electrical signals associated with sensation. In this way, electrodes 32 may be configured to deliver electroporation energy to tissue responsible for sensation during pacing, thereby irreversibly electroporating the tissue.

Lead 22 may deliver a set of stimulation pulses to the target site (902). For instance, lead 22 may be electrically coupled to stimulation device 14. Stimulation circuitry 52 of stimulation device 14 may provide electrical pulses to electrodes 32 based on a set of stimulation parameters stored in stimulation parameter repository 58. Electrodes 32 may measure a set of evoked response signals from the target site that is produced in response to the set of stimulation pulses (904). Processing circuitry 50 may determine a set of evoked response parameters based on the sensed set of evoked response signals and store the set of evoked response parameters in evoked response parameter repository 60.

Processing circuitry 50 may, via sensation detection module 56, determine a likelihood of sensation at the target site from a pacing therapy based on the set of stimulation parameters in stimulation parameter repository 58 and the set of evoked response parameters in evoked response parameter repository 60 (906). Responsive to processing circuitry 50 determining that there is no likelihood of sensation (or an acceptable level of risk) at the target site from the pacing therapy (“NO” branch of 906), lead 22 may be implanted at the target site (908). Responsive to processing circuitry 50 determining that there is a likelihood of sensation at the target site from the pacing therapy (“YES” branch of 906), electroporation device 62 may deliver electroporation energy via lead 22 to irreversibly electroporate tissue at the target site as described above (900).

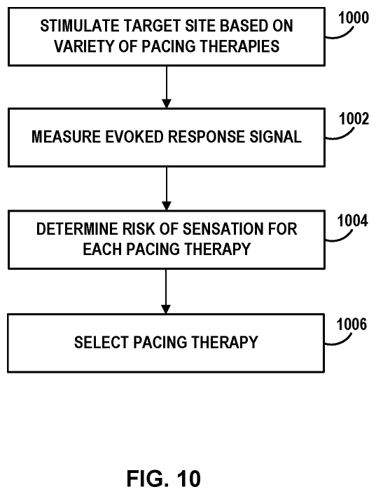

FIG. 10 is a flow diagram of an example technique for using system 10 in accordance with techniques of this disclosure. Lead 22 may deliver a set of stimulation pulses to the target site based on a variety of pacing therapies or configurations (e.g., pacing vectors, electrode combinations, pacing voltages, etc.) (1000). The variety of pacing therapies may be based on a set of stimulation parameters stored in stimulation parameter repository 58. Electrodes 32 may measure a set of evoked response signals from the target site that is produced in response to each of the pacing therapies (1002). Processing circuitry 50 may determine a set of evoked response parameters based on the sensed set of evoked response signals and store the set of evoked response parameters in evoked response parameter repository 60.

When executing sensation detection module 56, processing circuitry 50 may determine a likelihood of sensation at the target site from each pacing therapy and the set of evoked response parameters for the set of evoked response signals from the target site that is produced in response to each of the pacing therapies (1004). Processing circuitry 50 may select a pacing therapy based on the evoked response parameters (1006). For example, processing circuitry 50 may select the pacing therapy that has the highest pacing amplitude that does not result in detection of an evoked response (indicating a low likelihood of sensation). In some examples, processing circuitry 50 may select an alternative pacing therapy with a lower likelihood of sensation (e.g., a pacing therapy with a lower pacing amplitude) based on detection of an evoked response. In this way, the techniques may avoid or reduce sensation experienced by a patient during pacing therapy.

Various aspects of the disclosure have been described. These and other aspects are within the scope of the following claims.

Example 1. A stimulation device comprising: stimulation circuitry; sensing circuitry; and processing circuitry configured to: control the stimulation circuitry to deliver, based on a set of stimulation parameters, a set of stimulation pulses to a target site; control the sensing circuitry to sense a set of evoked response signals from the target site, wherein each evoked response signal of the set of evoked response signals is in response to a corresponding stimulation pulse from the set of stimulation pulses; measure a set of evoked response parameters for the set of evoked response signals; and determine, based on the set of stimulation parameters and the set of evoked response parameters, a likelihood of sensation at the target site from a pacing therapy.

Example 2. The stimulation device of Example 1, wherein the set of evoked response parameters comprises at least one of latency, morphology, frequency spectra, evoked response amplitude, or sensing vector.

Example 3. The stimulation device of Example 1 or 2, wherein the set of stimulation parameters comprises at least one of polarity, pulse width, pulse frequency, stimulation amplitude, or stimulation vector.

Example 4. The stimulation device of any one of Examples 1 to 3, wherein the processing circuitry is configured to determine the likelihood of sensation by determining whether an evoked response parameter of the set of evoked response parameters satisfies a corresponding evoked response parameter condition.

Example 5. The stimulation device of Example 4, wherein the processing circuitry is configured to determine whether the evoked response parameter satisfies the corresponding evoked response parameter condition by determining whether the evoked response parameter is equal to or greater than a corresponding evoked response parameter threshold.

Example 6. The stimulation device of any one of Examples 1 to 5, wherein the stimulation device is further configured to: configure electroporation energy to irreversibly electroporate tissue; and deliver the electroporation energy to the set of electrodes.

Example 7. The stimulation device of Example 6, wherein the stimulation device comprises an electroporation device.

Example 8. The stimulation device of any one of Examples 1 to 7, wherein the target site comprises at least one of muscle tissue or nerve tissue.

Example 9. The stimulation device of any one of Examples 1 to 8, further comprising a connector assembly configured to be coupled to an extracardiac elongated structure having one or more electrode.

Example 10. The stimulation device of any one of Examples 1 to 9, wherein the stimulation device comprises one of a pacemaker, an implantable cardioverter defibrillator, a cardiac resynchronization therapy device, or a neurostimulator.

Example 11. The stimulation device of any one of Examples 1 to 10, wherein the processing circuitry is further configured to transmit the likelihood of sensation.

Example 12. A system comprising: an extracardiac elongated structure configured to be navigated from an access point of a patient to a target site within a patient, wherein a distal portion of the elongated structure comprises a set of electrodes; and a stimulation device configured to be coupled to the extracardiac elongated structure, wherein the stimulation device comprises: stimulation circuitry; sensing circuitry; and processing circuitry configured to: control the stimulation circuitry to deliver, based on a set of stimulation parameters, a set of stimulation pulses to the target site; control the sensing circuitry to sense, based on a set of sensing parameters, a set of evoked response signals from the target site, wherein each evoked response signal of the set of evoked response signals is in response to a corresponding stimulation pulse from the set of stimulation pulses; measure a set of evoked response parameters for the set of evoked response signals; and determine, based on the set of stimulation parameters and the set of evoked response parameters, a likelihood of sensation at the target site from a pacing therapy.

Example 13. The system of Example 12, wherein the set of evoked response parameters comprises at least one of latency, morphology, frequency spectra, evoked response amplitude, or sensing vector.

Example 14. The system of Example 12 or 13, wherein the set of stimulation parameters comprises at least one of polarity, pulse width, pulse frequency, stimulation amplitude, or stimulation vector.

Example 15. The system of any one of Examples 12 to 14, wherein the processing circuitry is configured to determine the likelihood of sensation by determining whether an evoked response parameter of the set of evoked response parameters satisfies a corresponding evoked response parameter condition.

Example 16. The system of any one of Examples 12 to 15, wherein the processing circuitry is configured to determine whether the evoked response parameter satisfies the corresponding evoked response parameter condition by determining whether the evoked response parameter is equal to or greater than a corresponding evoked response parameter threshold.

Example 17. The system of any one of Examples 12 to 16, further comprising an electroporation device configured to: configure electroporation energy to irreversibly electroporate tissue; and deliver the electroporation energy to the set of electrodes.

Example 18. The system of any one of Examples 12 to 17, wherein the target site comprises at least one of muscle tissue or nerve tissue.

Example 19. The system of any one of Examples 12 to 18, wherein the extracardiac elongated structure is an introducer, an implant tool, or an implantable medical lead.

Example 20. The system of any one of Examples 12 to 19, further comprising an external electrode configured to be placed proximate a sternum of the patient.

Example 21. The system of any one of Examples 12 to 20, wherein the stimulation device further comprises a connector assembly configured to be coupled to the extracardiac elongated structure.

Example 22. The system of any one of Examples 12 to 21, wherein the stimulation device comprises one of a pacemaker, an implantable cardioverter defibrillator, a cardiac resynchronization therapy device, or a neurostimulator.

Example 23. The system of any one of Examples 12 to 22, wherein the processing circuitry is further configured to transmit the likelihood of sensation.

Example 24. A method comprising: delivering a set of stimulation pulses to the target site based on a set of stimulation parameters; sensing a set of evoked response signals from the target site, wherein each evoked response signal of the set of evoked response signals is in response to a corresponding stimulation pulse from the set of stimulation pulses; measuring a set of evoked response parameters for the set of evoked response signals; and determining, based on the set of stimulation parameters and the set of evoked response parameters, a likelihood of sensation at the target site from a pacing therapy.

Example 25. The method of Example 24, further comprising: responsive to determining that there is the likelihood of sensation at the target site from the pacing therapy, delivering electroporation energy to the target site; and after delivering the electroporation energy to the target site: delivering a current set of stimulation pulses to the target site based on the set of stimulation parameters; sensing, based on the set of sensing parameters, a current set of evoked response signals from the target site, wherein each evoked response signal of the current set of evoked response signals is in response to a corresponding stimulation pulse from the current set of stimulation pulses; measuring a current set of evoked response parameters for the current set of evoked response signals; and determining, based on the set of stimulation parameters and the current set of evoked response parameters, a current likelihood of sensation at the target site from the pacing therapy.

Example 26. The method of Example 24 or 25, wherein the set of evoked response parameters comprises at least one of latency, morphology, frequency spectra, evoked response amplitude, or sensing vector.

Example 27. The method of any one of Examples 24 to 26, wherein the set of stimulation parameters comprises at least one of polarity, pulse width, pulse frequency, stimulation amplitude, or stimulation vector.

Example 28. The method of any one of Examples 24 to 27, wherein determining the likelihood of sensation comprises determining whether an evoked response parameter of the set of evoked response parameters satisfies a corresponding evoked response parameter condition.

Example 29. The method of any one of Examples 24 to 28, wherein determining whether the evoked response parameter satisfies the corresponding evoked response parameter condition comprises determining whether the evoked response parameter is equal to or greater than a corresponding evoked response parameter threshold.

Example 30. The method of any one of Examples 24 to 29, further comprising: configuring electroporation energy to irreversibly electroporate tissue; and delivering the electroporation energy to the tissue.

Example 31. The method of any one of Examples 24 to 30, wherein the target site comprises at least one of muscle tissue or nerve tissue.

Example 32. The method of any one of Examples 24 to 31, further comprising transmitting the likelihood of sensation.

Example 33. A system comprising an extracardiac elongated structure configured to be navigated from an access point of a patient to a target site within a patient, wherein a distal portion of the elongated structure comprises a set of electrodes and the stimulation device of any one of Examples 1 to 11 and configured to be coupled to the extracardiac elongated structure.

Example 34. The system of Example 33, wherein the extracardiac elongated structure is an introducer, an implant tool, or an implantable medical lead.

Example 35. The system of any of claims 33 or 34, further comprising an external electrode configured to be placed proximate a sternum of the patient.

1. A stimulation device comprising:
stimulation circuitry;
sensing circuitry; and
processing circuitry configured to:
control the stimulation circuitry to deliver, based on a set of stimulation parameters, a set of stimulation pulses to a target site;
control the sensing circuitry to sense a set of evoked response signals from the target site, wherein each evoked response signal of the set of evoked response signals is in response to a corresponding stimulation pulse from the set of stimulation pulses;
measure a set of evoked response parameters for the set of evoked response signals; and
determine, based on the set of stimulation parameters and the set of evoked response parameters, a likelihood of sensation at the target site from a pacing therapy.

2. The stimulation device of claim 1, wherein the set of evoked response parameters comprises at least one of latency, morphology, frequency spectra, evoked response amplitude, or sensing vector.

3. The stimulation device of claim 1, wherein the set of stimulation parameters comprises at least one of polarity, pulse width, pulse frequency, stimulation amplitude, or stimulation vector.

4. The stimulation device of claim 1, wherein the processing circuitry is configured to determine the likelihood of sensation by determining whether an evoked response parameter of the set of evoked response parameters satisfies a corresponding evoked response parameter condition.

5. The stimulation device of claim 4, wherein the processing circuitry is configured to determine whether the evoked response parameter satisfies the corresponding evoked response parameter condition by determining whether the evoked response parameter is equal to or greater than a corresponding evoked response parameter threshold.

6. The stimulation device of claim 1,
wherein the stimulation device is further configured to:
configure electroporation energy to irreversibly electroporate tissue; and
deliver the electroporation energy to the set of electrodes.

7. The stimulation device of claim 6, wherein the stimulation device comprises an electroporation device.

8. The stimulation device of claim 1 wherein the target site comprises at least one of muscle tissue or nerve tissue.

9. The stimulation device of claim 1, further comprising a connector assembly configured to be coupled to an extracardiac elongated structure having one or more electrode.

10. The stimulation device of claim 1. wherein the stimulation device comprises one of a pacemaker, an implantable cardioverter defibrillator, a cardiac resynchronization therapy device, or a neurostimulator.

11. The stimulation device of claim 1. wherein the processing circuitry is further configured to transmit the likelihood of sensation.

12. A system comprising:
an extracardiac elongated structure configured to be navigated from an access point of a patient to a target site within a patient, wherein a distal portion of the elongated structure comprises a set of electrodes; and
a stimulation device configured to be coupled to the extracardiac elongated structure, wherein the stimulation device comprises:
stimulation circuitry;
sensing circuitry; and
processing circuitry configured to:
control the stimulation circuitry to deliver, based on a set of stimulation parameters, a set of stimulation pulses to the target site;
control the sensing circuitry to sense, based on a set of sensing parameters, a set of evoked response signals from the target site, wherein each evoked response signal of the set of evoked response signals is in response to a corresponding stimulation pulse from the set of stimulation pulses;
measure a set of evoked response parameters for the set of evoked response signals; and
determine, based on the set of stimulation parameters and the set of evoked response parameters, a likelihood of sensation at the target site from a pacing therapy.

13. The system of claim 12, wherein the extracardiac elongated structure is an introducer, an implant tool, or an implantable medical lead.

14. The system of claim 12, further comprising an external electrode configured to be placed proximate a sternum of the patient.

15. The system of claim 12, wherein the processing circuitry is configured to determine the likelihood of sensation by determining whether an evoked response parameter of the set of evoked response parameters satisfies a corresponding evoked response parameter condition.

16. The system of claim 12, further comprising an electroporation device configured to:
configure electroporation energy to irreversibly electroporate tissue; and
deliver the electroporation energy to the set of electrodes.

17. A method comprising:
delivering a set of stimulation pulses to the target site based on a set of stimulation parameters;
sensing a set of evoked response signals from the target site, wherein each evoked response signal of the set of evoked response signals is in response to a corresponding stimulation pulse from the set of stimulation pulses;
measuring a set of evoked response parameters for the set of evoked response signals; and
determining, based on the set of stimulation parameters and the set of evoked response parameters, a likelihood of sensation at the target site from a pacing therapy.

18. The method of claim 17, further comprising:
responsive to determining that there is the likelihood of sensation at the target site from the pacing therapy, delivering electroporation energy to the target site; and
after delivering the electroporation energy to the target site:
delivering a current set of stimulation pulses to the target site based on the set of stimulation parameters;
sensing, based on the set of sensing parameters, a current set of evoked response signals from the target site, wherein each evoked response signal of the current set of evoked response signals is in response to a corresponding stimulation pulse from the current set of stimulation pulses;
measuring a current set of evoked response parameters for the current set of evoked response signals; and
determining, based on the set of stimulation parameters and the current set of evoked response parameters, a current likelihood of sensation at the target site from the pacing therapy.

19. The method of claim 17, wherein determining the likelihood of sensation comprises determining whether an evoked response parameter of the set of evoked response parameters satisfies a corresponding evoked response parameter condition.

20. The method of claim 17, further comprising:
configuring electroporation energy to irreversibly electroporate tissue; and
delivering the electroporation energy to the tissue.
